(19) United States
(12) Patent Application Publication
(10) Pub. No.: US 2025/0181911 A1
(43) Pub. Date: Jun. 5, 2025

(54) METHOD OF PRE-PROCESSING TRAINING DATA FOR MOLECULAR DYNAMICS SIMULATION AND APPARATUS FOR PERFORMING THE METHOD

(71) Applicant: SAMSUNG ELECTRONICS CO., LTD., SUWON-SI (KR)

(72) Inventors: Geonu Kim, Suwon-si (KR); Byunggook Na, Suwon-si (KR); Gunhee Kim, Suwon-si (KR); Yongdeok Kim, Suwon-si (KR); Hyuntae Cho, Suwon-si (KR); Seung jin Kang, Suwon-si (KR); Heejae Kim, Suwon-si (KR)

(21) Appl. No.: 18/932,812

(22) Filed: Oct. 31, 2024

(30) Foreign Application Priority Data
Dec. 5, 2023 (KR) 10-2023-0174204

(51) Int. Cl. G06N 3/08; G06N 3/042; G06N 3/047

(52) U.S. Cl. CPC G06N 3/08; G06N 3/042; G06N 3/047

(57) ABSTRACT

Provided is a method of pre-processing training data for a molecular dynamics simulation. The method includes obtaining geometric information of a molecule that includes a plurality of atoms, identifying a set of edges between the plurality of atoms in the molecule based on the geometric information, filtering the set of edges using a probability function based on the geometric information to obtain a filtered set of edges, and generating a training set for a graph neural network (GNN) including a graph of the molecule based on the filtered set of edges.

### CROSS-REFERENCE TO RELATED APPLICATIONS

This application is based on and claims priority under 35 U.S.C. § 119 to Korean Patent Application No. 10-2023-0174204, filed on Dec. 5, 2023, in the Korean Intellectual Property Office, the contents of which are incorporated by reference herein in their entirety.

### BACKGROUND

### 1. Field of the Invention

One or more embodiments relate to a method of pre-processing training data for training an interaction potential model used in a molecular dynamics simulation and an apparatus for performing the method.

### 2. Description of the Related Art

Molecular dynamics simulation deals with prediction of atomic behavior that is based on the quality and relevance of training data that parameterize the underlying force fields. In some cases, the process of preparing the training data may involve labor-intensive manual curation and selection of molecular structures. However, such molecular structures may be prone to biases and time constraints.

In some cases, existing methods may not be able to adequately capture the diverse and dynamic nature of molecular systems, leading to suboptimal performance and limited applicability in complex scenarios. Therefore, there is a need in the art for systems and methods that can accurately capture the properties of the molecular structures while minimizing the computational resources.

### SUMMARY

The present disclosure describes systems and methods for pre-processing training data of a neural network for a molecular dynamics simulation using a graph neural network (GNN). An embodiment of the present disclosure includes a method for pre-processing training data to reduce the complexity of a graph that represents a structure of a molecular system. For example, the graph of the molecular structure may be represented using edges of an atom with the surrounding atoms. In some cases, the pre-processing may include sampling an edge based on applying a probability function to the edge. In some examples, the edge may be formed by considering characteristics (e.g., a distance, etc.) between atoms.

According to an aspect, there is provided a method of pre-processing training data for a molecular dynamics simulation, the method including obtaining geometric information of a molecule that includes a plurality of atoms, identifying a set of edges between the plurality of atoms in the molecule based on the geometric information, filtering the set of edges using a probability function based on the geometric information to obtain a filtered set of edges, and generating a training set for a graph neural network (GNN) including a graph of the molecule based on the filtered set of edges.

The geometric information may include distance information between the plurality of atoms in the molecule.

The probability function comprises:

$$u ( x ; {R}_{hard} ) = \{ \begin{matrix}1. \cdot if \cdot x \leq {R}_{hard} \\ 0. … otherwise\end{matrix} . , .. where \cdot {R}_{hard} < {R}_{cut} . \qquad [ Equation ]$$

wherein u(x; R_{hard}) may denote a probability that an atom, x, is sampled based on an R_{hard} condition, R_{hard} may denote a definite sampling radius, and R_{cut} may denote an edge cutoff radius.

The probability function comprises:

$$p ( x ; {R}_{hard} ) = \{ \begin{matrix}1. … if \cdot x \leq {R}_{hard} \\ \frac{{R}_{cut} - \cdot x}{{R}_{cut} - {R}_{hard}} \cdot otherwise\end{matrix} . , .. where \cdot {R}_{hard} < {R}_{cut} . \qquad [ Equation ]$$

wherein p(x; R_{hard}) may denote a probability that an atom, x, is sampled based on an R_{hard} condition, R_{hard} may denote a definite sampling radius, and R_{cut} may denote an edge cutoff radius.

The graph including the formed edges based on the probability function may be used to train a graph neural network (GNN). In some cases, the GNN may be trained using the training set.

The GNN may include one of machine-learning interatomic potential (MLIP) and a machine-learning force field (MLFF).

According to another aspect, there is provided a method of performing a molecular dynamics simulation by a prediction apparatus, the method including obtaining a geometric information of a molecule that includes a plurality of atoms, generating a graph including a plurality of edges among the plurality of atoms in the molecule, and generating, using a graph neural network (GNN), a simulation result for the molecule based on the graph, in which the GNN is trained using a training set including a training graph, wherein a set of edges of the training graph is filtered based on a probability function.

The simulation result may include at least one of potential energy information, stress information, physical force information, or charge information on the structure of the molecule.

The generating of the graph by forming the edge of the molecule may include selecting at least a portion of the sampled edge in the molecule based on the probability function.

According to still another aspect, there is provided an apparatus for pre-processing training data, the apparatus including one or more processors, a memory, and one or more programs stored in the memory and executed by the one or more processors, in which the one or more processors are configured to obtain geometric information of a molecule that includes a plurality of atoms, identify a set of edges between the plurality of atoms in the molecule based on the geometric information, filter the set of edges based on the geometric information to obtain a filtered set of edges, and generate a training set for a graph neural network (GNN) including a graph of the molecule based on the filtered set of edges.

According to an aspect, there is provided a training method including obtaining training data including a set of edges among a plurality of atoms in a molecule, filtering the set of edges using a probability function based on geometric information of the molecule to obtain filtered training data, and training a graph neural network (GNN) using the filtered training data.

In some aspects, training the GNN comprises computing a simulation result based on the filtered training data, computing a loss function based on the simulation result, and updating parameters of the GNN based on the loss function.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings:

FIG. 1 illustrates a method of forming an edge around each atom in an input molecular structure, according to an embodiment;

FIG. 2 illustrates an example in which training data pre-processed through an apparatus is used, according to an embodiment;

FIG. 3 is a flowchart illustrating a method of pre-processing training data for a molecular dynamics simulation by an apparatus, according to an embodiment;

FIGS. 4A and 4B illustrate an example of a probability function, according to an embodiment;

FIG. 5 is a block diagram illustrating an apparatus for pre-processing training data, according to an embodiment;

FIG. 6 is a flowchart illustrating a method of performing a molecular dynamics simulation through a trained model, according to an embodiment; and

FIGS. 7A and 7B are graphs illustrating the training performance of training data, according to an embodiment.

FIG. 8 is a flowchart illustrating a method of generating a training set for a GNN, according to an embodiment.

FIG. 9 is a flowchart illustrating a method of training a GNN, according to an embodiment.

### DETAILED DESCRIPTION

The present disclosure describes systems and methods for pre-processing training data of a neural network for a molecular dynamics simulation using a graph neural network (GNN). An embodiment of the present disclosure includes a method for pre-processing training data to reduce the complexity of a graph that represents a structure of a molecular system. For example, the graph of the molecular structure may be represented using edges of an atom with the surrounding atoms. In some cases, the pre-processing may include sampling an edge based on applying a probability function to the edge. In some examples, the edge may be formed by considering characteristics (e.g., a distance, etc.) between atoms.

In some cases, performing a molecular dynamic simulation may include labor-intensive manual curation and selection of molecular structures, prone to biases and time constraints. Conventionally used methods may struggle to adequately capture the diverse and dynamic nature of molecular systems, leading to suboptimal performance and limited applicability in complex scenarios. Additionally, in some cases, when constructing a complex graph based molecular structure, the amount of computations and training time of the model significantly increase. Moreover, the accuracy and generalization ability of a trained model may decrease and technical integration with existing dynamics simulation software may be difficult when a limited number of surrounding atoms are used for the simulation process.

Accordingly, embodiments of the present disclosure include a method of forming a complex graph based on a graph neural network (GNN)-based MLIP model. In some cases, the MLIP model may be configured to form edges with surrounding atoms of a molecule included within a radius, e.g., a cutoff radius. In some cases, each atom of the molecule may be used as a central atom. In some cases, edges may be formed between each atom (i.e., central atom) of the molecule and atoms surrounding the central atom and located within the cutoff radius. An embodiment of the disclosure includes a method for selecting edges formed within the cutoff radius based on a probability function.

In some cases, machine-learning interatomic potential (MLIP) may utilize machine learning techniques to approximate the potential energy surface of atomic interactions in materials. For example, MLIP may be used for predicting a complex interaction between atoms with high accuracy at the level of quantum mechanics. In some cases, an MLIP model may be used to generate potential energy, atomic force, or stress values for an atomic structure provided as input to the MLIP model. In some examples, the MLIP model may use a feature engineering method. For example, the feature engineering method may compute physical features representing the surrounding environment of each atom for an input atomic structure followed by training the MLIP model. In some cases, the MLIP model may be trained based on providing the physical features as input to a multi-layer perceptron model.

Accordingly, by using an MLIP model, embodiments of the present disclosure are able to overcome the inaccuracies of existing molecular dynamics models while achieving high accuracy at the quantum mechanics level. In some cases, use of the MLIP model along with pre-processed input data enables prediction of a complex interaction between atoms with high accuracy. Additionally, by using the said combination of the MLIP model with pre-processed input data, the high calculation cost of existing quantum mechanics may be effectively reduced while enabling a computational simulation of a large-scale system.

Embodiments of the present disclosure include generating training data that is pre-processed based on generating a graph. In some cases, a structure with different types of atomic configuration may be received as an input of a molecule for performing a molecular dynamics simulation. Graph neural network (GNN)-based MLIP models show high accuracy and implement a computation process by representing the surrounding environment as a graph for each atom. According to an embodiment, the GNN-based MLIP model may consider atoms as nodes and may receive, as an input, a graph that forms an edge, based on position information and the radius of the atoms. In some cases, a portion of the formed edges may be selected based on a probability function that considers distance between the atoms.

Accordingly, by implementing an edge sampling or selecting method based on the probability function, embodiments of the present disclosure are able to generate graphs with different structures for the same molecular structure. Additionally, the probability-based sampling method may select a short length edge with a high probability and select a long length edge with a low probability. In some cases, the edge with a short length may be used more in the overall training process and an edge with a long length may be used less in the training process. Therefore, a GNN-based model may be efficiently trained to have a good performance in a short time using less computational resources.

The present disclosure describes systems and methods of pre-processing training data for a molecular dynamic simulation. Embodiments of the present disclosure include obtaining geometric information of a molecule that includes a plurality of atoms. In some cases, a set of edges may be identified between the plurality of atoms in the molecule based on the geometric information. Next, the set of edges are filtered using a probability function based on the geometric information to obtain a filtered set of edges and a training set for a graph neural network (GNN) may be generated. In some cases, the training set for the GNN may include a graph of the molecule based on the filtered set of edges.

An embodiment of the present disclosure includes a method of performing a molecular dynamics simulation. In some cases, the method comprises obtaining a geometric information of a molecule that includes a plurality of atoms. A graph may be generated, wherein the graph includes a plurality of edges among the plurality of atoms in the molecule. A simulation result for the molecule may be generated using the GNN. In some cases, the simulation result may be generated based on the graph. In some cases, the GNN is trained using a training set including a training graph, wherein a set of edges of the training graph are filtered based on a probability function.

An embodiment of the present disclosure includes obtaining training data comprising a set of edges among a plurality of atoms in a molecule. In some cases, the set of edges may be filtered using a probability function based on geometric information of the molecule to obtain filtered training data. In some cases, a GNN may be trained using the filtered training data.

Accordingly, by leveraging advanced techniques from machine learning and data processing, embodiments of the present disclosure are able to automate the generation, selection, and augmentation of training data, streamlining the preparation process and enhancing the quality and diversity of datasets used in molecular dynamics simulations. Additionally, embodiments enable researchers to expedite the generation of training data and improve the accuracy and reliability of molecular dynamics simulations for complex molecular systems.

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. As used herein, the singular forms “a”, “an”, and “the” include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

Also, in the description of the components, terms such as first, second, A, B, (a), (b) or the like may be used herein when describing components of the present disclosure. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” to the constituent elements.

The same name may be used to describe an element included in the embodiments described above and an element having a common function. Unless otherwise mentioned, the descriptions of the embodiments may be applicable to the following embodiments and thus, duplicated descriptions will be omitted for conciseness.

Embodiments of the present disclosure include a method to obtain a highly accurate molecular dynamics simulation result. In some examples, a machine-learning interatomic potential (MLIP) model may be trained with high accuracy. One or more embodiments include a method of forming a complex graph considering a large number of atoms. In some cases, the graph may be generated based on atomic position information of given training data or based on a large amount of training data and training an MLIP model.

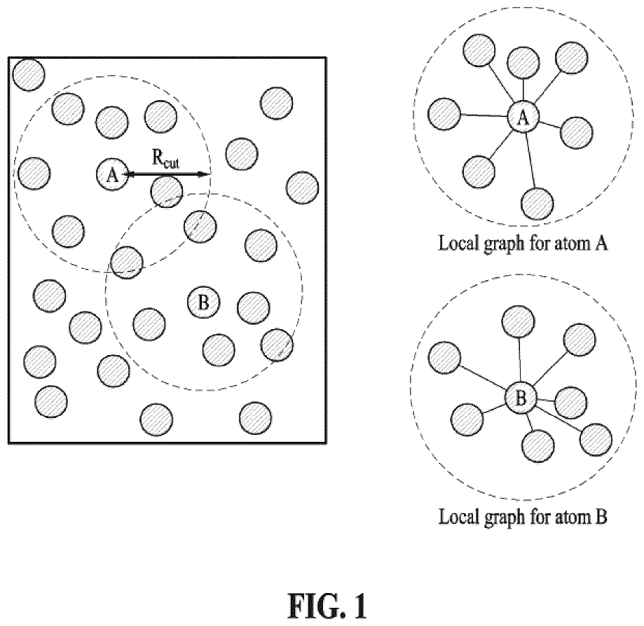

FIG. 1 illustrates a method of forming an edge around each atom in an input molecular structure, according to an embodiment.

FIG. 1 shows a method of forming a complex graph for a structure given as input data based on a cutoff radius. In some cases, a graph neural network (GNN)-based MLIP model may include a method of configuring a graph by forming edges of each atom with surrounding elements included within a radius R_{cut} to meet a condition. As shown with reference to FIG. 1, each atom may be used as a central atom.

In some cases, a Graph Neural Network (GNN) is a type of neural network designed to analyze and process graph-structured data. Unlike traditional neural networks, which operate on grid-like data such as images or sequences, GNNs can model relationships and dependencies between entities represented as nodes and edges in a graph. By iteratively aggregating information from neighboring nodes, GNNs can learn to extract features and make predictions based on the graph's topology. Accordingly, GNNs may be well-suited for tasks such as node classification, link prediction, and graph-level regression, with applications spanning social networks, recommendation systems, and biological networks.

A Machine Learning Interatomic Potential (MLIP) model is a computational method that utilizes machine learning techniques to approximate the potential energy surface of atomic interactions in materials. MLIP models are trained on large datasets of atomistic simulations or quantum mechanical calculations to learn the relationship between atomic configurations and their corresponding energies. By leveraging techniques such as neural networks or kernel methods, MLIP models can accurately predict the energy of atomic configurations with high efficiency, enabling the study of material properties, phase transitions, and chemical reactions at scales and timeframes inaccessible to traditional methods. The MLIP models may be used in materials science, chemistry, and condensed matter physics for accelerating the exploration and design of new materials with tailored properties and functionalities.

In some cases, a method of forming a graph structure may be based on, e.g., a physical fact that atoms close to the central atom have the maximum influence on each other. Additionally, the model training and computational efficiency may vary depending on the size of the radius R_{cut}.

In some cases, as the radius R_{cut} increases, the accuracy of a model may generally increase since the influence of surrounding atoms at a far distance is considered. Accordingly, the amount of computations and training time of the model may increase significantly since the number of edges may increase and the graph structure may become complex.

In some cases, the number of surrounding atoms close to each other may be limited in a ranking-based manner which alleviates the complexity. As such, the accuracy and generalization ability of a trained model may decrease and technical integration with dynamics simulation software may be difficult.

Accordingly, the present disclosure describes a method of selecting edges that may be formed within the radius R_{cut} based on a probability function.

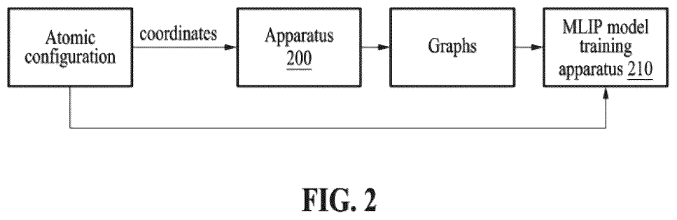

FIG. 2 illustrates an example in which training data pre-processed through an apparatus is used, according to an embodiment.

An apparatus 200 may receive a molecular structure (e.g., atomic configuration) as an input and generate a graph. For example, the apparatus 200 may exist separately from an MLIP model training apparatus 210 based on a GNN and may operate independently. In some examples, the apparatus 200 may operate on the same hardware or a different hardware.

Hereinafter, description of a molecule may not be limited to the dictionary scope, i.e., the characteristics of a molecule as used herein extend beyond mere linguistic definitions, encompassing a broader range of attributes and behaviors.

For example, a structure with various types of atomic configurations may be received as an input of a molecule for performing a molecular dynamics simulation.

In some cases, the GNN-based MLIP model may consider atoms as nodes and may receive, as an input, a graph that forms an edge, based on position information and the radius of the atoms.

Hereinafter, a method of forming an edge considering geometric information, such as distance information, is described. Additionally, a method of including and training only a portion of one or more edges included within the radius may be described. In some cases, a decrease in the accuracy of a model trained through the MLIP model training apparatus 210 may be minimized.

The present disclosure uses the MLIP model training apparatus 210 for illustrative purposes and embodiments are not limited thereto. For example, any other complex GNN-based models may be trained using the graph of the molecular structure generated from the apparatus 200 and the molecular dynamics simulation result corresponding to the molecular structure.

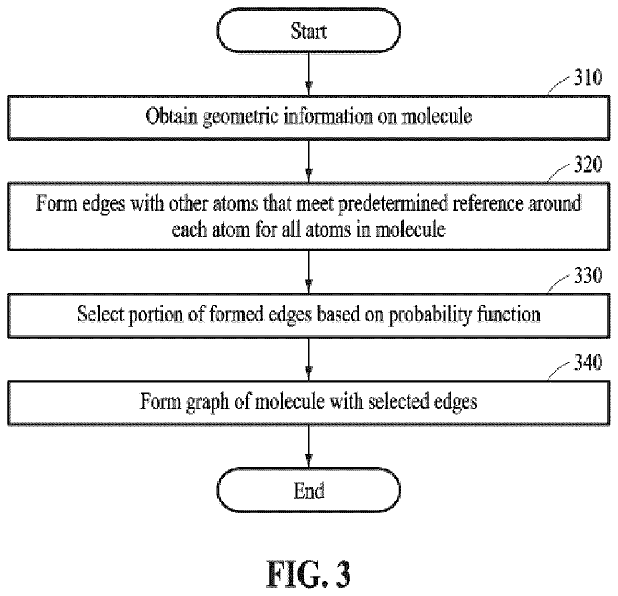

FIG. 3 is a flowchart illustrating a method of pre-processing training data for a molecular dynamics simulation by an apparatus, according to an embodiment.

Operations to be described hereinafter may be performed sequentially. However, embodiments may not be necessarily limited thereto and for example, the order of the operations may change and at least two of the operations may be performed in parallel.

An apparatus for pre-processing training data for a potential model used in a molecular dynamics simulation (hereinafter, referred to as an ‘apparatus’) may pre-process training data through operations 310 to 340.

In operation 310, the apparatus may obtain geometric information on a molecule.

In some cases, the apparatus may obtain the geometric information on a molecule to pre-process training data. For example, data including position information of atoms in a three-dimensional (3D) molecule may be obtained. The data may be expressed using Equation 1 below.

$$S = \{ {x}_{i} ∈ {R}^{3} \vert 1 \leq i \leq n \} \qquad [ Equation 1 ]$$

Here, n denotes the number of atoms, i denotes an atomic index, and R³ denotes a 3D real number value.

In operation 320, the apparatus may form edges with other atoms that meet a predetermined reference around each atom for the plurality of atoms in the molecule based on the geometric information.

Using the cutoff radius described with reference to FIG. 1, edges may be formed with atoms included within the cutoff radius having the same size for each atom included in the molecule. The formed edges may be expressed using Equation 2 below.

$$E = \{ e = ( {x}_{i} , {x}_{j} ) \vert \cdot \vert e \vert \leq {R}_{cut} , i \neq j \} \text{?} \qquad [ Equation 2 ]$$

$$\text{?} \text{indicates text missing or illegible when filed}$$

Here, e denotes an edge, xᵢ and xⱼ denote indexes of two atoms connected to the edge, and R_{cut} denotes a cutoff radius.

In operation 330, the apparatus may select a portion of the formed edges based on a probability function.

In some cases, at least a portion of the edges may be selected by applying the probability function to the formed edges. In some examples, only at least a portion of the edges may be selected based on the probability function to the formed edges. The probability function may be expressed using Equation 3 below.

$$f ( e ; \text{?} p ( \vert e \vert ) ) \qquad [ Equation 3 ]$$

$$\text{?} \text{indicates text missing or illegible when filed}$$

Here, p(|e|) denotes a sampling probability. Hereinafter, the probability function may be designed differently based on considering distance information. For example, when the value of Equation 3 is 1, the edge e may be selected. Additionally or alternatively, when the value obtained using Equation 3 is 0, the edge e may not be selected.

In some cases, an edge may be selected by applying the corresponding probability function to select an edge at each training step. In some cases, graphs with different structures may be formed because edges selected at each sampling are different even for the same molecular structure. In some cases, graphs with different structures may be formed since a sampling method is based on the probability function. In some cases, an edge with a short length may have a high probability of being selected since the sampling probability is designed to have a characteristic of monotonically decreasing according to distance. In some cases, the edge with a short length may be used more in the overall training process which may imply a high influence of the central atom on surrounding atoms in case of, e.g., a physical feature of shorter distances. Simultaneously, an edge with a long length corresponding to the radius may have a low probability of being selected. In some cases, a GNN-based model may be efficiently trained for a good performance in a short time through less computations since the edge with a long length may be included in the training process.

In operation 340, the apparatus may form a graph of a molecule with the selected edges.

In some cases, the apparatus may output the graph of the molecule using the edges formed through operations 320 and 330 when information including 3D atomic position information is obtained.

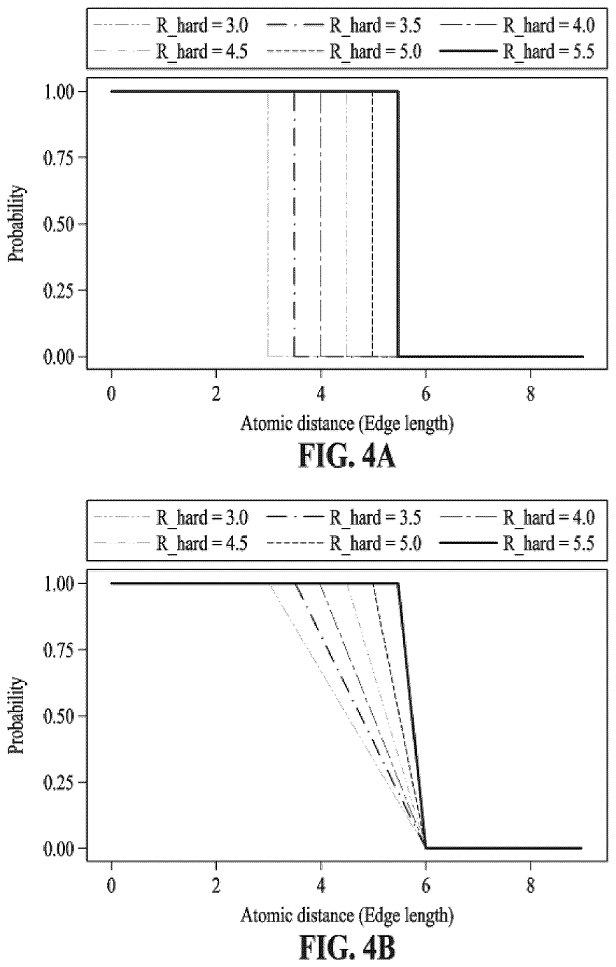

FIGS. 4A and 4B illustrate an example of a probability function, according to an embodiment.

As shown in FIG. 4A, a deterministic sampling function may be used. Additionally, as shown in FIG. 4B, a probabilistic sampling function may be used.

The deterministic sampling function shown in FIG. 4A may use a unit step function and may be expressed using Equation 4 below.

$$u ( x ; {R}_{hard} ) = \{ \begin{matrix}1. \cdot if \cdot x \leq {R}_{hard} \\ 0. … otherwise\end{matrix} . , .. where \cdot {R}_{hard} < {R}_{cut} . \qquad [ Equation 4 ]$$

Here, R_{hard} denotes a definite sampling radius and may be determined in a radius size less than or equal to R_{cut}. In some cases, an edge with a distance greater than or equal to R_{hard} may not be selected using R_{hard}. For example, according to FIG. 4A, the same sampling result may be obtained for the same molecular structure.

FIG. 4B may be expressed as Equation 5 below.

$$[ Equation 5 ]$$

$$p ( x ; {R}_{hard} ) = \{ \begin{matrix}1. … if \cdot x \leq {R}_{hard} \\ \frac{{R}_{cut} - \cdot x}{{R}_{cut} - {R}_{hard}} \cdot otherwise\end{matrix} . , .. where \cdot {R}_{hard} < {R}_{cut} .$$

According to Equation 5, an edge with a length equal to or shorter than R_{hard} may be selected. In some cases, an edge exceeding R_{hard} may be selected stochastically. As a result, different graphs may be formed for the same molecular structure.

Accordingly, an effect similar to data augmentation may be obtained since given data is trained in various ways. In some cases, the generalization performance of a model may be improved. Therefore, the generalization performance of an MLIP model may be enhanced in which the prediction result accuracy is used in the extrapolation region. Additionally, in some cases, a new simulation may be used to produce a more accurate result for a new molecular structure that the MLIP model does not experience during training.

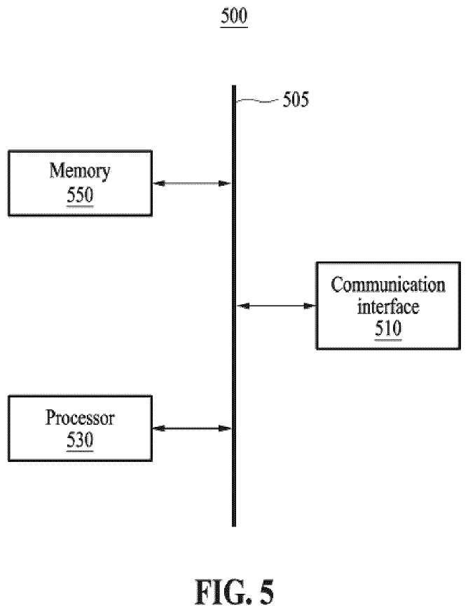

FIG. 5 is a block diagram illustrating an apparatus for pre-processing training data, according to an embodiment.

Referring to FIG. 5, a prediction apparatus 500 may include a communication interface 510, a processor 530, and a memory 550. The communication interface 510, the processor 530, and the memory 550 may communicate with each other via a communication bus 505.

In some cases, the communication interface 510 may receive a molecular structure.

The processor 530 may generate a graph for the molecular structure received by the communication interface 510. The processor 530 may form an edge based on a probability function in the molecular structure and generate the graph based on the edge.

The memory 550 may store a variety of information generated by the processing (implemented by the processor 530) described with reference to FIGS. 1-4. Additionally, the memory 550 may store a variety of data and programs. The memory 550 may include a volatile memory or a non-volatile memory. The memory 550 may include a high-capacity storage medium such as a hard disk to store a variety of data.

Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause at least one processor of processor 510 to perform various functions described herein.

In some cases, memory 550 includes a basic input/output system (BIOS) that controls basic hardware or software operations, such as an interaction with peripheral components or devices. In some cases, memory 550 includes a memory controller that operates memory cells of memory 550. For example, the memory controller may include a row decoder, column decoder, or both. In some cases, memory cells within memory 550 store information in the form of a logical state.

Additionally, the processor 530 may perform at least one method described with reference to FIGS. 1 to 4 or an algorithm corresponding to the at least one method. The processor 530 may be a hardware-implemented data processing device having a circuit that is physically structured to execute desired operations. For example, the desired operations may include code or instructions included in a program. The processor 530 may be implemented as, for example, a central processing unit (CPU), a graphics processing unit (GPU), or a neural processing unit (NPU). The prediction apparatus 500 that is hardware-implemented may include, for example, a microprocessor, a CPU, a processor core, a multi-core processor, a multiprocessor, an application-specific integrated circuit (ASIC), and a field-programmable gate array (FPGA).

In some cases, processor 530 is configured to operate a memory array using a memory controller. In other cases, a memory controller is integrated into processor 530. In some cases, processor 530 is configured to execute computer-readable instructions stored in memory 550 to perform various functions. In some aspects, processor 530 includes special purpose components for modem processing, baseband processing, digital signal processing, or transmission processing. According to some aspects, processor 530 comprises the one or more processors described herein. The processor 530 may execute the program and control the prediction apparatus 500. Code of the program executed by the processor 530 may be stored in the memory 550.

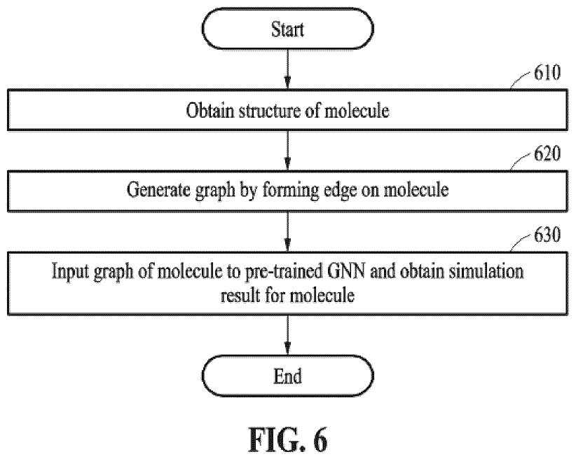

FIG. 6 is a flowchart illustrating a method of performing a molecular dynamics simulation via a trained model, according to an embodiment.

In some cases, a prediction apparatus may be used for performing a molecular dynamics simulation. In some cases, the prediction apparatus may include a neural network model trained using training data that is pre-processed based on the methods described with reference to FIGS. 1 to 5.

In operation 610, the prediction apparatus may obtain a structure of a molecule. In some cases, the prediction apparatus may obtain a geometric information of a molecule that includes a plurality of atoms.

The prediction apparatus may obtain data including 3D position information of atoms in the molecule. For example, the prediction apparatus may obtain 3D position information of atoms in the molecule for the molecular dynamics simulation.

In operation 620, the prediction apparatus may generate a graph by forming an edge on the molecule. For example, the prediction apparatus may generate a graph including a plurality of edges among the plurality of atoms in the molecule.

In some examples, the prediction apparatus may form edges with atoms included in the radius having the same size for each atom. For example, the prediction apparatus may form edges with an atom included in the molecule for performing the molecular dynamics simulation.

According to an example, at least a portion of the formed edges may be selected based on the probability function (as described with reference to FIGS. 3-4). Additionally or alternatively, the edge may be formed by sampling the edge of an input atomic structure.

The graph for the molecule may be generated based on the formed edges.

In operation 630, the prediction apparatus may input the graph of the molecule to a pre-trained GNN and obtain the simulation result for the molecule. In some cases, the prediction apparatus may use the GNN to generate a simulation result for the molecule based on the graph. In some cases, the GNN is trained using a training set including a training graph, wherein a set of edges of the training graph is filtered based on a probability function.

For example, the GNN (i.e., pre-trained GNN) may include a neural network trained using the graph of the molecule in which an edge of each atom is sampled based on the probability function. In some examples, the pre-trained GNN may include a neural network trained based on training data that is pre-processed (as described with reference to FIGS. 1 to 5).

According to an example, the simulation result may include at least one of potential energy information, stress information, physical force information, or charge information on the structure of the molecule.

The method described herein may be used in various fields to which the molecular dynamics simulation may be applied, such as a process simulation of a semiconductor device, battery, or pharmacology.

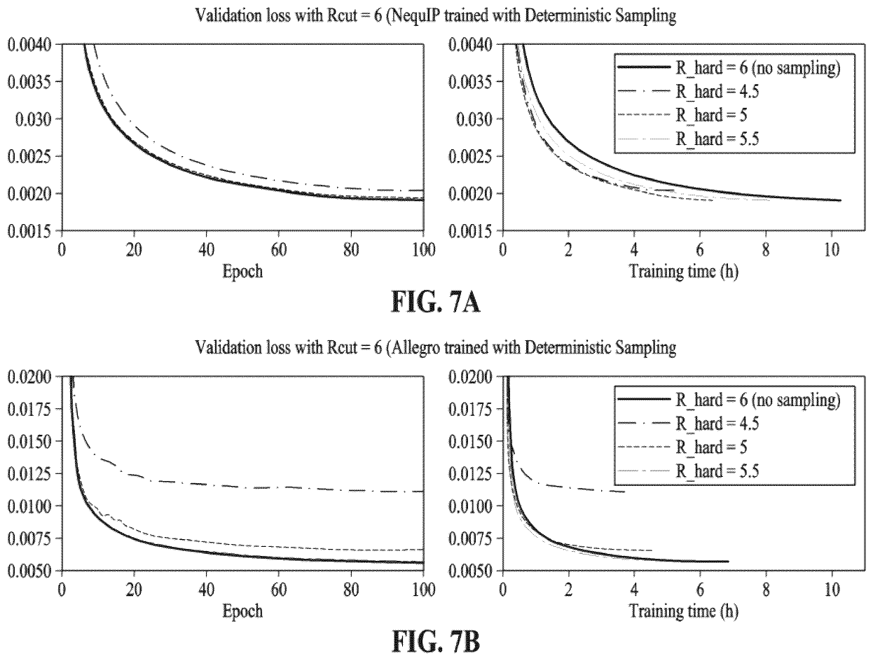

FIGS. 7A and 7B are graphs illustrating the training performance of training data, according to an embodiment.

FIGS. 7A and 7B illustrate experimental results of applying sampling using a probability function to HfO (Hafnium Oxide) data set training of NequIP and Allegro as an example of an MLIP model. In some examples, the HfO dataset may consist of, but not limited thereto, data related to Hafnium Oxide, which may include properties, structures, compositions, etc.

According to an exemplary embodiment, NequIP and Allegro may be neural network models or algorithms trained for tasks, such as material property prediction, molecular simulation, or data analysis. In some cases, Neural Quantum Interaction Predictor (NequIP) specializes in predicting quantum interactions within molecular systems, enabling accurate simulations of chemical reactions and material properties. The NequIP model leverages deep learning techniques to model quantum interactions efficiently, making it invaluable for computational chemistry and materials science research. Additionally, Allegro is a neural network framework optimized for handling large-scale datasets and conducting high-dimensional data analysis. Allegro includes a robust architecture that enables rapid processing and extraction of insights from diverse datasets, thereby enabling powerful data analysis capabilities.

For example, when an MLIP model operating at R_{cut}=6 is trained, two pieces of probability sampling may be applied to dataset training. In some examples, eight V100 GPUs may be used and 100 epochs for training may be performed in the training experiments.

FIG. 7A shows experimental result of applying a deterministic sampling function to training. For example, each model may be visualized as epoch and training time. According to FIG. 7A, when NequIP is trained with R_{hard}≥5, NequIP may reach the accuracy of the trained result without sampling. Additionally, according to FIG. 7B, when Allegro is trained with R_{hard}≥5.5, Allegro may reach the accuracy of the trained result without sampling.

Considering the training speed, R_{hard}={4.5, 5, and 5.5} may show a result that is approximately 2 times, 1.6 times, and 1.3 times faster, respectively.

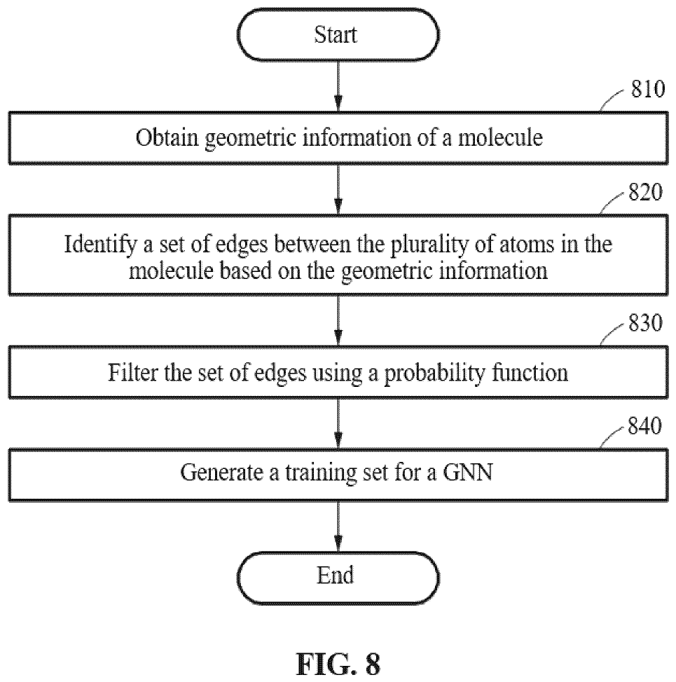

FIG. 8 is a flowchart illustrating a method of generating training data for a molecular dynamics simulation, according to an embodiment.

Operations to be described hereinafter may be performed sequentially. However, embodiments may not be necessarily limited thereto and for example, the order of the operations may change and at least two of the operations may be performed in parallel.

A method for generating training data for a potential model used in a molecular dynamics simulation may generate training data based on operations 810 to 840.

In operation 810, the method may include obtaining geometric information of a molecule. In some cases, the molecule may include a plurality of atoms.

In some examples, the obtained geometric information of the molecule may include position information of atoms in a three-dimensional (3D) molecule.

In operation 820, the method may include identifying a set of edges among the plurality of atoms. In some cases, the set of edges may be identified for each atom in the molecule, e.g., based on the geometric information received at operation 810. In some examples, the set of edges identified may satisfy a predetermined distance requirement. For example, the identified edges may be generated around each atom for the plurality of atoms in the molecule based on the geometric information. Further detail regarding the identification of edges are described with reference to FIG. 3, and repeated descriptions are omitted herein for brevity.

In operation 830, the method may include filtering the set of edges using a probability function based on the geometric information to obtain a filtered set of edges. For example, at least a portion of the edges may be selected by applying the probability function to the formed edges. In some examples, only at least a portion of the edges may be selected based on the probability function to the formed edges.

In some cases, an edge may be selected by applying a probability function at each training step. In some cases, an edge with a short length may have a high probability of being selected since the sampling probability is designed to have a characteristic of monotonically decreasing according to distance. In some cases, the edge with a short length may be used more in the overall training process which may imply a (e.g., a physical feature of) high influence of the central atom on surrounding atoms in case of shorter distances. Simultaneously, an edge with a long length corresponding to the radius may have a low probability of being selected. Further details regarding the filtering of the set of edges have been provided with reference to FIGS. 3-4.

In operation 840, the method may include generating a training set for a graph neural network (GNN). In some cases, the GNN may include a graph of the molecule based on the filtered set of edges. For example, the GNN may form a graph of a molecule with the filtered edges. In some cases, the method may generate a training dataset for the GNN, wherein the GNN includes a graph of the molecule using the edges formed through operations 820 and 830 based on geometric information (e.g., including 3D atomic position) is obtained in operation 810.

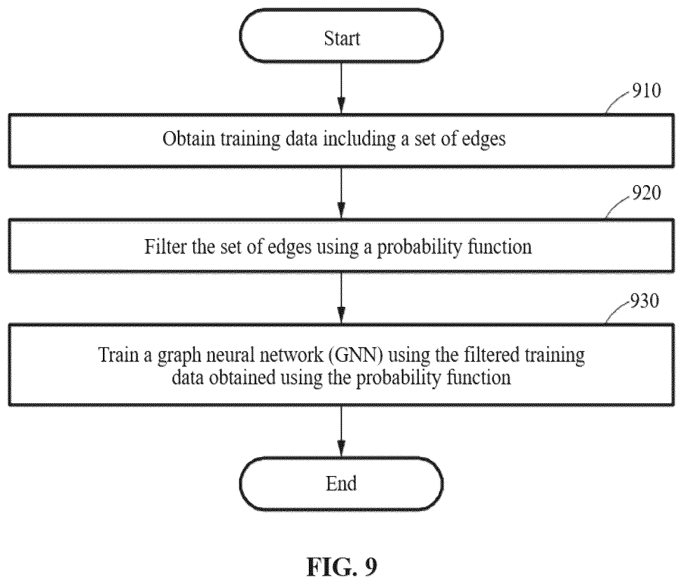

FIG. 9 is a flowchart illustrating a training method, according to an embodiment.

Operations to be described hereinafter may be performed sequentially. However, embodiments may not be necessarily limited thereto and for example, the order of the operations may change and at least two of the operations may be performed in parallel.

A method for training a graph neural network may be described based on operations 910 to 930. Further details regarding each of operations 910-930 are provided with reference to FIGS. 3-4.

In operation 910, the method may include obtaining training data including a set of edges among a plurality of atoms in a molecule. In some cases, the plurality of atoms in the molecule may be arranged as a three-dimensional (3D) structure. In some cases, the method may include obtaining position information of the atoms in the three-dimensional (3D) structure of the molecule.

In some cases, the method may include obtaining training data based on identifying a set of edges among the plurality of atoms. In some cases, the training data comprising the set of edges may be identified for each atom in the molecule, e.g., based on the position information of the atoms. In some examples, the training data comprising the set of identified edges may satisfy a predetermined distance requirement. e.g., the edges may be generated around each atom in the molecule based on the distance requirement.

In operation 920, the method may include filtering the set of edges using a probability function based on the geometric information to obtain filtered training data. For example, at least a portion of the edges may be selected by applying the probability function to the edges (i.e., training data obtained in operation 910).

In some cases, filtered training data comprising selected edges may be obtained by applying a probability function at each training step. In some cases, filtered training data may include an edge with a short length. For example, an edge with a short length may have a high probability of being selected. In some cases, the edge with a short length may be used more in the overall training process which may imply a (e.g., a physical feature of) high influence of the central atom on surrounding atoms in case of shorter distances. Simultaneously, an edge with a long length corresponding to the radius may have a low probability of being selected. Further details regarding the filtering of the set of edges have been provided with reference to FIGS. 3-4.

In operation 930, the method may include training a graph neural network (GNN) using the filtered training data. In some cases, the GNN may include a graph of the molecule generated based on the filtered training data. In some cases, the filtered training data may be obtained by applying the probability function to the training data (as described in operation 920 and with reference to FIGS. 3-4). For example, the GNN may form a graph of a molecule with the filtered training data comprising select edges from the set of identified edges. In some cases, the method may generate a training dataset for the GNN, wherein the GNN is trained using filtered training data generated through operations 910 and 920.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described embodiments, or vice versa.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

A number of embodiments have been described above. Nevertheless, it should be understood that various modifications may be made to these embodiments. For example, suitable results may be achieved if the described techniques are performed in a different order and/or if components in a described system, architecture, device, or circuit are combined in a different manner and/or replaced or supplemented by other components or their equivalents.

The processes discussed above are intended to be illustrative and not limiting. One skilled in the art would appreciate that the steps of the processes discussed herein may be omitted, modified, combined, and/or rearranged, and any additional steps may be performed without departing from the scope of the invention. More generally, the above disclosure is meant to be exemplary and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

Accordingly, other implementations are within the scope of the following claims.

What is claimed is:

1. A method comprising:
obtaining geometric information of a molecule that includes a plurality of atoms;
identifying a set of edges among the plurality of atoms in the molecule based on the geometric information;
filtering the set of edges using a probability function based on the geometric information to obtain a filtered set of edges; and
generating a training set for a graph neural network (GNN) including a graph of the molecule based on the filtered set of edges.

2. The method of claim 1, wherein the geometric information comprises distance information between the plurality of atoms in the molecule.

3. The method of claim 1, wherein the probability function comprises:
$$u ( x ; {R}_{hard} ) = \{ \begin{matrix}1. \cdot if \cdot x \leq {R}_{hard} \\ 0. … otherwise\end{matrix} . , .. where \cdot {R}_{hard} < {R}_{cut} \text{?} \qquad [ Equation ]$$
$$\text{?} \text{indicates text missing or illegible when filed}$$
wherein u(x; R_{hard}) denotes a probability that an atom, x, is sampled based on an R_{hard} condition, R_{hard} denotes a definite sampling radius, and R_{cut} denotes an edge cutoff radius.

4. The method of claim 1, wherein the probability function comprises:
$$p ( x ; {R}_{hard} ) = \{ \begin{matrix}1. … if \cdot x \leq {R}_{hard} \\ \frac{{R}_{cut} - \cdot x}{{R}_{cut} - {R}_{hard}} \cdot otherwise\end{matrix} . , .. where \cdot {R}_{hard} < {R}_{cut} . \qquad [ Equation ]$$
wherein p(x; R_{hard}) denotes a probability that an atom, x, is sampled based on an R_{hard} condition, R_{hard} denotes a definite sampling radius, and R_{cut} denotes an edge cutoff radius.

5. The method of claim 1, further comprising:
training the GNN using the training set.

6. The method of claim 5, wherein the GNN comprises one of machine-learning interatomic potential (MLIP) and a machine-learning force field (MLFF).

7. A method comprising:
obtaining a geometric information of a molecule that includes a plurality of atoms;
generating a graph including a plurality of edges among the plurality of atoms in the molecule;
generating, using a graph neural network (GNN), a simulation result for the molecule based on the graph, wherein the GNN is trained using a training set including a training graph, wherein a set of edges of the training graph is filtered based on a probability function.

8. The method of claim 7, wherein the simulation result comprises at least one of potential energy information, stress information, physical force information, or charge information on the structure of the molecule.

9. The method of claim 7, wherein the generating of the graph by forming the edge of the molecule comprises selecting at least a portion of the sampled edge in the molecule based on the probability function.

10. The method of claim 7, wherein the probability function comprises:
$$u ( x ; {R}_{hard} ) = \{ \begin{matrix}1. \cdot if \cdot x \leq {R}_{hard} \\ 0. … otherwise\end{matrix} . , .. where \cdot {R}_{hard} < {R}_{cut} \text{?} \qquad [ Equation ]$$
$$\text{?} \text{indicates text missing or illegible when filed}$$
wherein u(x;R_{hard}) denotes a probability that an atom, x, is sampled based on an R_{hard} condition, R_{hard} denotes a definite sampling radius, and R_{cut} denotes an edge cutoff radius.

11. The method of claim 7, wherein the probability function comprises:
$$p ( x ; {R}_{hard} ) = \{ \begin{matrix}1. … if \cdot x \leq {R}_{hard} \\ \frac{{R}_{cut} - \cdot x}{{R}_{cut} - {R}_{hard}} \cdot otherwise\end{matrix} . , .. where \cdot {R}_{hard} < {R}_{cut} . \qquad [ Equation ]$$
wherein p(x;R_{hard}) denotes a probability that an atom, x, is sampled based on an R_{hard} condition, R_{hard} denotes a definite sampling radius, and R_{cut} denotes an edge cutoff radius.

12. A non-transitory computer-readable storage medium storing instructions that, when executed by a processor, cause the processor to perform the method of claim 1.

13. An apparatus for pre-processing training data, the apparatus comprising:
one or more processors;
a memory; and
one or more programs stored in the memory and executed by the one or more processors,
wherein the one or more processors are configured to:
obtain geometric information of a molecule that includes a plurality of atoms;
identify a set of edges between the plurality of atoms in the molecule based on the geometric information;
filter the set of edges using a probability function based on the geometric information to obtain a filtered set of edges; and
generate a training set for a graph neural network (GNN) including a graph of the molecule based on the filtered set of edges.

14. The apparatus of claim 13, wherein the geometric information comprises distance information between the plurality of atoms in the molecule.

15. The apparatus of claim 13, wherein the probability function comprises:
$$u ( x ; {R}_{hard} ) = \{ \begin{matrix}1. \cdot if \cdot x \leq {R}_{hard} \\ 0. … otherwise\end{matrix} . , .. where \cdot {R}_{hard} < {R}_{cut} . \qquad [ Equation ]$$
wherein u(x; R_{hard}) denotes a probability that an atom, x, is sampled based on an R_{hard} condition, R_{hard} denotes a definite sampling radius, and R_{cut} denotes an edge cutoff radius.

16. The apparatus of claim 13, wherein the probability function comprises:
$$p ( x ; {R}_{hard} ) = \{ \begin{matrix}1. … if \cdot x \leq {R}_{hard} \\ \frac{{R}_{cut} - \cdot x}{{R}_{cut} - {R}_{hard}} \cdot otherwise\end{matrix} . , .. where \cdot {R}_{hard} < {R}_{cut} . \qquad [ Equation ]$$
wherein p(x;R_{hard}) denotes a probability that an atom, x, is sampled based on an R_{hard} condition, R_{hard} denotes a definite sampling radius, and R_{cut} denotes an edge cutoff radius.

17. The apparatus of claim 13, further comprising:
training the GNN using the training set.

18. The apparatus of claim 17, wherein the GNN comprises one of machine-learning interatomic potential (MLIP) and a machine-learning force field (MLFF).

19. A method comprising:
obtaining training data including a set of edges among a plurality of atoms in a molecule;
filtering the set of edges using a probability function based on geometric information of the molecule to obtain filtered training data; and
training a graph neural network (GNN) using the filtered training data.

20. The method of claim 19, wherein training the GNN comprises:
computing a simulation result based on the filtered training data;
computing a loss function based on the simulation result; and
updating parameters of the GNN based on the loss function.
